(19) United States
(12) Patent Application Publication
(10) Pub. No.: US 2026/0224915 A1
(43) Pub. Date: Aug. 6, 2026

(54) PATIENT IMMOBILIZATION

(71) Applicant: LEO CANCER CARE, INC., Middleton, WI (US)

(72) Inventor: Michael K. Bauer, Bothell, WA (US)

(21) Appl. No.: 19/146,601

(22) PCT Filed: Jan. 11, 2024
(86) PCT No.: PCT/US2024/011151

§ 371 Date: Jul. 9, 2025

Related U.S. Application Data
(60) Provisional application No. 63/438,978, filed on Jan. 13, 2023.

(51) Int. Cl. A61N 5/10

(52) U.S. Cl. CPC A61N 5/1049; A61N 2005/1061

(57) ABSTRACT

Provided herein is technology relating to medical diagnosis and treatment and particularly, but not exclusively, to a patient support (a non-integrated patient support) that is provided apart from a medical imaging or radiotherapy apparatus and that is a duplicate of a patient support that is used for medical imaging and/or radiotherapy (an integrated patient support). The non-integrated patient support is used to determine a patient posture and/or a patient support immobilization configuration for subsequent use when positioning the patient for diagnosis and/or treatment using an integrated patient support that is a component of a medical diagnosis and treatment system.

This application claims benefit of United States provisional patent application Ser. No. 63/438,978, filed Jan. 13, 2023, which is incorporated herein by reference in its entirety.

### FIELD

Provided herein is technology relating to medical diagnosis and treatment and particularly, but not exclusively, to a patient support (a non integrated patient support) that is provided apart from a medical imaging or radiotherapy apparatus and that is a duplicate of a patient support that is used for medical imaging and/or radiotherapy (an integrated patient support). The non integrated patient support is used to determine a patient posture and/or a patient support immobilization configuration for subsequent use when positioning the patient for diagnosis and/or treatment using an integrated patient support that is a component of a medical diagnosis and treatment system.

Methods of the technology comprise positioning a patient on the isolated, non-integrated patient support and recording a patient support configuration to provide the recorded patient support configuration for subsequent use. Systems of the technology comprise the non-integrated patient support and a second patient support that is an integrated part of a medical imaging and/or radiotherapy system. Data describing a patient support configuration recorded on the non-integrated patient support are used subsequently to configure the integrated patient support for medical imaging and/or radiotherapy of the patient according to the recorded patient support configuration.

### BACKGROUND

Conventional radiation therapy of a patient typically comprises a simulation phase, a treatment planning phase, and a treatment phase. During the simulation phase, a patient is positioned on a patient support in a treatment position, patient immobilization devices are produced, and images of the patient are recorded (e.g., using computerized tomography (CT)). During the treatment planning phase, the previously recorded images are used to produce a treatment plan for the patient. During the treatment phase, the patient is positioned on a patient support using the immobilization devices and the patient is exposed to radiation according to the treatment plan. Thus, during conventional radiation therapy, imaging is performed using a medical imaging device (e.g., CT scanner) that is separate from the radiation therapy apparatus (e.g., comprising a linac for producing therapeutic radiation). Some recent advances in radiation therapy technology provide a system comprising the medical imaging components and the radiotherapy components in a single installed system that further comprises a patient support that is used for both imaging and therapy tasks. Accordingly, in these recent radiation therapy technologies, patient imaging and patient treatment are performed in the same location using the same patient support, which increases the accuracy and reproducibility of treatment.

However, while one patient is being imaged or treated, the patient support is not available for determining a patient position and producing immobilization devices and configurations for other patients.

### SUMMARY

Accordingly, provided herein is technology relating to an isolated, non-integrated patient support that is provided apart from a medical imaging or radiotherapy apparatus, and related systems and methods. Patients may be positioned on the non-integrated patient support to acquire and record a patient support configuration while an integrated patient support of a medical imaging or radiotherapy system is used for medical imaging or radiotherapy of another patient.

For example, in some embodiments, the technology provides a medical therapy system comprising a subsystem comprising a first patient support (an integrated patient support): and a second patient support (a non-integrated patient support). In some embodiments, the first patient support is structured to support a patient in an upright position and the second patient support is structured to support the patient in the upright position. In some embodiments, the upright position is a standing, sitting, kneeling, or perched position. In some embodiments, the subsystem is a medical imaging subsystem comprising an imaging source and a detector. In some embodiments, the subsystem is a radiotherapy subsystem comprising a radiotherapy source. In some embodiments, the subsystem is a medical imaging and radiotherapy subsystem comprising an imaging source, a detector, and a radiotherapy source. In some embodiments, the radiotherapy source is a static source. In some embodiments, the first patient support is configured to rotate around a substantially vertical axis. In some embodiments, the first patient support is coupled to components that translate the first patient support in three dimensions and that rotate the first patient support around three axes. In some embodiments, the second patient support comprises a stand or base. In some embodiments, the second patient support is mounted to a floor. In some embodiments, the first patient support and second patient support are in different rooms. In some embodiments, the medical therapy system further comprises a database. In some embodiments, the medical therapy system further comprises a computer. In some embodiments, the database comprises a data structure describing a patient support configuration comprising a number of index values recorded from the second patient support. In some embodiments, the database further comprises patient information for a patient and wherein the patient information is associated with a patient support configuration. In some embodiments, the first patient support is configured according to the patient support configuration for the patient. In some embodiments, the patient is identified or associated with the patient information. In some embodiments, the first patient support comprises a seat pan, a shin rest, and a heel stop; and wherein the second patient support comprises a seat pan, a shin rest, and a heel stop. In some embodiments, the first patient support further comprises a back rest; and wherein the second patient support further comprises a back rest. In some embodiments, the index values comprise a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value.

Further embodiments relate to methods. For example, in some embodiments, methods comprise providing a medical therapy system comprising a first patient support (an integrated patient support) and a second patient support (a non integrated patient support); configuring the second patient support to provide a configured second patient support; recording a patient support configuration describing the configuration of the configured second patient support to provide a recorded patient support configuration; and configuring the first patient support according to the recorded patient support configuration. In some embodiments, methods further comprise positioning a patient on the configured second patient support to provide a configured second patient support comprising a positioned patient. In some embodiments, methods further comprise checking the configuration of the configured second patient support. In some embodiments, methods further comprise adjusting the configuration of the configured second patient support. In some embodiments, recording a patient support configuration comprises recording a number of index values describing the configuration of the configured second patient support. In some embodiments, the recorded patient support configuration comprises a number of index values describing the configuration of the configured second patient support. In some embodiments, the index values comprise a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value. In some embodiments, methods further comprise positioning a patient on the first patient support and performing medical imaging and/or radiotherapy on the patient.

In some embodiments of the methods provided, the first patient support is structured to support a patient in an upright position and the second patient support is structured to support the patient in the upright position. In some embodiments, the upright position is a standing, sitting, kneeling, or perched position. In some embodiments, a medical imaging subsystem comprises the first patient support, an imaging source, and a detector. In some embodiments, a radiotherapy subsystem comprises the first patient support and a radiotherapy source. In some embodiments, a medical imaging and radiotherapy subsystem comprises the first patient support, an imaging source, a detector, and a radiotherapy source. In some embodiments, the radiotherapy source is a static source. In some embodiments, the first patient support is configured to rotate around a substantially vertical axis. In some embodiments, the first patient support is coupled to components that translate the first patient support in three dimensions and that rotate the first patient support around three axes. In some embodiments, the second patient support comprises a stand or base. In some embodiments, the second patient support is mounted to a floor. In some embodiments, the first patient support and second patient support are in different rooms.

In some embodiments, recording a patient support configuration comprises producing a data structure in a database describing the patient support configuration.

In some embodiments, recording a patient support configuration further comprises recording patient information for a patient associated with the patient support configuration. In some embodiments, the patient is identified or associated with the patient information.

In some embodiments of methods, the first patient support comprises a seat pan, a shin rest, and a heel stop; and wherein the second patient support comprises a seat pan, a shin rest, and a heel stop. In some embodiments, the first patient support further comprises a back rest; and wherein the second patient support further comprises a back rest.

In some embodiments, the technology relates to use of a medical therapy system comprising a first patient support and a second patient support to record a patient support configuration and provide the patent support configuration for medical imaging and radiotherapy of a patient. In some embodiments, the second patient support is used to record the patient support configuration and the recorded patient support configuration is used to configure the first patent support. Further embodiments relate to use of a medical therapy system as described herein for medical imaging and radiotherapy of a patient. Related embodiments provide use of a method as described herein for medical imaging and radiotherapy of a patient.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

Embodiments of the technology may also relate to an apparatus for performing the operations herein. This apparatus may be specially constructed for the required purposes and/or it may comprise a general-purpose computing device selectively activated or reconfigured by a computer program stored in the computer. Such a computer program may be stored in a non transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings.

FIG. 1A is a perspective view of a patient support.

FIG. 1B is a side view of a patient support.

FIG. 1C is a side view of a patient support on which a patient is positioned.

FIG. 1D is a side view of a patient support showing exemplary adjustable heights and angles of some of the components of the patient support.

FIG. 2A is a schematic drawing of a system comprising a non integrated patient support and an integrated patient support that is used for medical imaging.

FIG. 2B is a schematic drawing of a system comprising a non-integrated patient support and an integrated patient support that is used for medical radiotherapy.

FIG. 2C is a schematic drawing of a system comprising a non integrated patient support and an integrated patient support that is used for both medical imaging and medical radiotherapy FIG. 2D is a schematic drawing of a system comprising a non integrated patient support, an integrated patient support, and a database.

FIG. 2E is a schematic drawing of a system comprising a non-integrated patient support, an integrated patient support, a power supply, a computer, and a database.

FIG. 3 is a flowchart for a method of configuring a non integrated patient support and recording a patient support configuration.

FIG. 4 is a flowchart for a method of configuring an integrated patient support according to a recorded patient support configuration.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to an isolated, non integrated patient support that is provided apart from a medical imaging or radiotherapy apparatus. The technology also provides methods related to the isolated, non-integrated patient support and systems comprising the isolated, non integrated patient support. For example, in some embodiments, the isolated, non integrated patient support finds use in methods comprising positioning a patient and recording a patient support configuration to provide a recorded patient support configuration for subsequent use in positioning a patient for medical imaging and/or radiotherapy. In some embodiments, the technology relates to a medical imaging and radiotherapy system comprising a first patient support and a second patient support, wherein the first patient support is an isolated, non-integrated patient support and the first patient support is used to record a patient support configuration and thus provide a recorded patient support configuration; the second patient support is an integrated part of a medical imaging and/or radiotherapy system; and the recorded patient support configuration is used to configure the second patient support for medical imaging of the patient and/or for radiotherapy of the patient. In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X. free” as used herein means “without X”, where X is a feature of the technology 8 omitted in the “X free” technology. For example, a “calcium free” composition does not comprise calcium, a “mixing-free” method does not comprise a mixing step, etc.

Although the terms “first”, “second”, “third”, etc. may be used herein to describe various steps, elements, compositions, components. regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre determined threshold; conversely, when an entity is said to be “absent”, it means the level or amount of this entity is below a pre determined threshold. The pre determined threshold is the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control.

Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor. a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object oriented programming language to communicate with a computer system. However, the program(s) can be implemented in assembly or machine language, if desired. In any case, the language is a compiled or interpreted language, and combined with hardware implementations. In some embodiments, a “system” comprises or is divisible into a plurality of smaller systems, which may be referred to as “subsystems” (e.g., a first subsystem, a second subsystem, . . . , an nth subsystem).

As used herein, the term “computed tomography” is abbreviated “CT” and refers both to tomographic and non tomographic radiography. For instance, the term “CT” refers to numerous forms of CT, including but not limited to X-ray CT, positron emission tomography (PET), single-photon emission computed tomography (SPECT), and photon counting computed tomography. Generally, computed tomography (CT) comprises use of an X-ray source and a detector that rotates around a patient and subsequent reconstruction of images into different planes. In embodiments of CT (e.g., devices, apparatuses, and methods provided for CT) described herein, the X-ray source is a static source and the patient is rotated with respect to the static source. Currents for X-rays used in CT describe the current flow from a cathode to an anode and are typically measured in milliamperes (mA).

As used herein, the term “structured to [verb]” means that the identified element or assembly has a structure that is shaped, sized, disposed, coupled, and/or configured to perform the identified verb. For example, a member that is “structured to move” is movably coupled to another element and includes elements that cause the member to move or the member is otherwise configured to move in response to other elements or assemblies. As such, as used herein, “structured to [verb]” recites structure and not function. Further, as used herein, “structured to [verb]” means that the identified element or assembly is intended to, and is designed to, perform the identified verb.

As used herein, the term “associated” means that the elements are part of the same assembly and/or operate together or act upon/with each other in some manner. For example, an automobile has four tires and four hub caps. While all the elements are coupled as part of the automobile, it is understood that each hubcap is “associated” with a specific tire.

As used herein, the term “coupled” refers to two or more components that are secured, by any suitable means, together. Accordingly, in some embodiments, the statement that two or more parts or components are “coupled” shall mean that the parts are joined or operate together either directly or indirectly, e.g., through one or more intermediate parts or components. As used herein, “directly coupled” means that two elements are directly in contact with each other. As used herein, “fixedly coupled” or “fixed” or “mounted” means that two components are coupled so as to move as one while maintaining a constant orientation relative to each other. Accordingly, when two elements are coupled, all portions of those elements are coupled. A description, however, of a specific portion of a first element being coupled to a second element, e.g., an axle first end being coupled to a first wheel, means that the specific portion of the first element is disposed closer to the second element than the other portions thereof.

Further, an object resting on another object held in place only by gravity is not “coupled” to the lower object unless the upper object is otherwise maintained substantially in place. That is, for example, a book on a table is not coupled thereto, but a book glued to a table is coupled thereto.

As used herein, the term “removably coupled” or “temporarily coupled” means that one component is coupled with another component in an essentially temporary manner. That is, the two components are coupled in such a way that the joining or separation of the components is easy and does not damage the components. Accordingly, “removably coupled” components is readily uncoupled and recoupled without damage to the components.

As used herein, the term “operatively coupled” means that a number of elements or assemblies, each of which is movable between a first position and a second position, or a first configuration and a second configuration, are coupled so that as the first element moves from one position/configuration to the other, the second element moves between positions/configurations as well. It is noted that a first element is “operatively coupled” to another without the opposite being true.

As used herein, the term “rotatably coupled” refers to two or more components that are coupled in a manner such that at least one of the components is rotatable with respect to the other.

As used herein, the term “translatably coupled” refers to two or more components that are coupled in a manner such that at least one of the components is translatable with respect to the other.

As used herein, the term “temporarily disposed” means that a first element or assembly is resting on a second element or assembly in a manner that allows the first element/assembly to be moved without having to decouple or otherwise manipulate the first element. For example, a book simply resting on a table, e.g., the book is not glued or fastened to the table, is “temporarily disposed” on the table.

As used herein, the term “correspond” indicates that two structural components are sized and shaped to be similar to each other and is coupled with a minimum amount of friction. Thus, an opening which “corresponds” to a member is sized slightly larger than the member so that the member may pass through the opening with a minimum amount of friction. This definition is modified if the two components are to fit “snugly” together. In that situation, the difference between the size of the components is even smaller whereby the amount of friction increases. If the element defining the opening and/or the component inserted into the opening are made from a deformable or compressible material, the opening may even be slightly smaller than the component being inserted into the opening. With regard to surfaces, shapes, and lines. two, or more, “corresponding” surfaces, shapes, or lines have generally the same size, shape, and contours.

As used herein, a “path of travel” or “path,” when used in association with an element that moves, includes the space an element moves through when in motion. As such, any element that moves inherently has a “path of travel” or “path.”

As used herein, the statement that two or more parts or components “engage” one another shall mean that the elements exert a force or bias against one another either directly or through one or more intermediate elements or components. Further, as used herein with regard to moving parts, a moving part may “engage” another element during the motion from one position to another and/or may “engage” another element once in the described position. Thus, it is understood that the statements, “when element A moves to element A first position, element A engages element B,” and “when element A is in element A first position, element A engages element B” are equivalent statements and mean that element A either engages element B while moving to element A first position and/or element A either engages element B while in element A first position.

As used herein, the term “operatively engage” means “engage and move.” That is, “operatively engage” when used in relation to a first component that is structured to move a movable or rotatable second component means that the first component applies a force sufficient to cause the second component to move. For example, a screwdriver is placed into contact with a screw. When no force is applied to the screwdriver, the screwdriver is merely “coupled” to the screw. If an axial force is applied to the screwdriver, the screwdriver is pressed against the screw and “engages” the screw.

However, when a rotational force is applied to the screwdriver, the screwdriver “operatively engages” the screw and causes the screw to rotate. Further, with electronic components, “operatively engage” means that one component controls another component by a control signal or current.

As used herein, the term “number” shall mean one or an integer greater than one (e.g., a plurality).

As used herein, in the phrase “[x]moves between its first position and second position,” or, “[y] is structured to move [x] between its first position and second position,” “[x]” is the name of an element or assembly. Further, when [x] is an element or assembly that moves between a number of positions, the pronoun “its” means “[x],” i.e., the named element or assembly that precedes the pronoun “its.”

As used herein, a “radial side/surface” for a circular or cylindrical body is a side/surface that extends about, or encircles, the center thereof or a height line passing through the center thereof. As used herein, an “axial side/surface” for a circular or cylindrical body is a side that extends in a plane extending generally perpendicular to a height line passing through the center. That is, generally, for a cylindrical soup can, the “radial side/surface” is the generally circular sidewall and the “axial side(s)/surface(s)” are the top and bottom of the soup can.

As used herein, a “diagnostic” test includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject having a disease or condition or the likelihood that such a subject will respond favorably to a compound (e.g., a pharmaceutical, e.g., a drug) or other treatment.

As used herein, the term “condition” refers generally to a disease, malady, injury, event, or change in health status.

As used herein, the term “treating” or “treatment” with respect to a condition refers to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof. In some embodiments, “treatment” comprises exposing a patient or a portion thereof (e.g., a tissue, organ, body part, or other localize region of a patient body) to radiation (e.g., electromagnetic radiation, ionizing radiation).

As used herein, the term “beam” refers to a stream of radiation (e.g., electromagnetic wave and/or or particle radiation). In some embodiments, the beam is produced by a source and is restricted to a small-solid angle. In some embodiments, the beam is collimated. In some embodiments, the beam is generally unidirectional. In some embodiments, the beam is divergent.

As used herein, the term “patient” or “subject” refers to a mammalian animal that is identified and/or selected for imaging and/or treatment with radiation.

Accordingly, in some embodiments, a patient or subject is contacted with a beam of radiation, e.g., a primary beam produced by a radiation source. In some embodiments, the patient or subject is a human. In some embodiments, the patient or subject is a veterinary or farm animal, a domestic animal or pet, or animal used for clinical research. In some embodiments, the subject or patient has cancer and/or the subject or patient has either been recognized as having or at risk of having cancer.

As used herein, the term “treatment volume” or “imaging volume” refers to the volume (e.g., tissue) of a patient that is selected for imaging and/or treatment with radiation. For example, in some embodiments, the “treatment volume” or “imaging volume” comprises a tumor in a patient (e.g., a cancer patient). As used herein, the term “healthy tissue” refers to the volume (e.g., tissue) of a patient that is not and/or does not comprise the treatment volume. In some embodiments, the imaging volume is larger than the treatment volume and comprises the treatment volume.

As used herein, the term “radiation source” or “source” refers to an apparatus that produces radiation (e.g., ionizing radiation in the form of photons (e.g., described as particles or waves)). In some embodiments, a radiation source is a linear accelerator (“linac”) that produces radiation in the form of x-rays or electrons to treat a patient by contacting a treatment volume of the patient with the x-ray or electron beam. In some embodiments, the source produces radiation in the form of particles (e.g., photons, electrons, neutrons, hadrons, ions (e.g., protons, carbon ions, other heavy ions)). In some embodiments, the source produces radiation in the form of electromagnetic waves (e.g., x rays and gamma rays having a wavelength in the range of approximately 1 pm to approximately 1 nm).

A “source” may produce radiation (e.g., x-rays) for imaging (an “imaging source”) or for radiotherapy (a “radiotherapy source” or “therapy source”). In typical use, an imaging source produces radiation having energies in the range of 1 to 1000 kV (e.g., a “kilovolt source”) and a radiotherapy source produces energies in the range of 1 to 1000 MV (e.g., a “megavolt source”). However, the technology is not limited to imaging using a kilovolt source and is not limited to radiotherapy using a megavolt source; and thus the technology includes imaging and radiotherapy modes and components using energies known in the art for use in imaging and radiotherapy (e.g., kilovolt (kV) or megavolt (MV) imaging, kV or MV CT, MV and lower dose (e.g., kV) radiotherapy technologies, and other imaging and therapy technologies of any energy). While it is understood that radiation can be described as having both wave-like and particle-like aspects, it is sometimes convenient to refer to radiation in terms of waves and sometimes convenient to refer to radiation in terms of particles. Accordingly, both descriptions are used throughout without limiting the technology and with an understanding that the laws of quantum mechanics provide that every particle or quantum entity is described as either a particle or a wave.

As used herein, the term “static source” refers to a source that does not revolve around a patient during use of the source for imaging or therapy. In particular, a “static source” remains fixed with respect to an axis passing through the patient while the patient is being imaged or treated. While the patient may rotate around said axis to produce relative motion between the static source and rotating patient that is equivalent to the relative motion of a source revolving around a static patient, a static source does not move with reference to a third object, frame of reference (e.g., a treatment room in which a patient is positioned), or patient axis of rotation during imaging or treatment, while the patient is rotated with respect to said third object, said frame of reference (e.g., said treatment room in which said patient is positioned), or patient axis of rotation through the patient during imaging or treatment. A static source may be installed on a mobile platform and the static source may move with respect to the Earth and fixtures on the Earth as the mobile platform moves to transport the static source. Thus, the term “static source” may refer to a mobile “static source” provided that the mobile “static source” does not revolve around an axis of rotation through the patient during imaging or treatment of the patient. Further, the static source may translate and/or revolve around the patient to position the static source prior to imaging or treatment of the patient or after imaging or treatment of the patient. Thus, the term “static source” may refer to a source that translates or revolves around the patient in non-imaging and non-treatment use, e.g., to position the source relative to the patient when the patient is not being imaged and/or treated. In some embodiments, the “static source” is a photon source and thus is referred to as a “static photon source”.

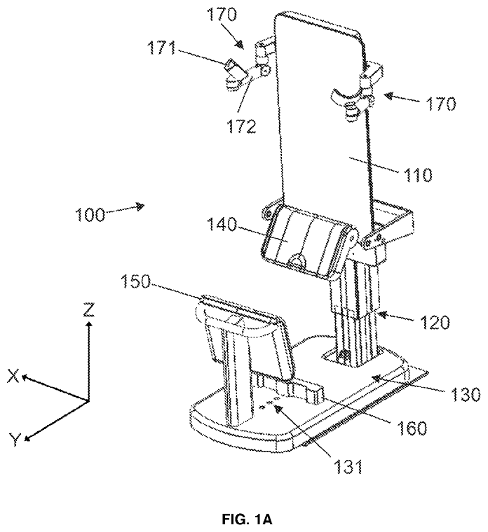

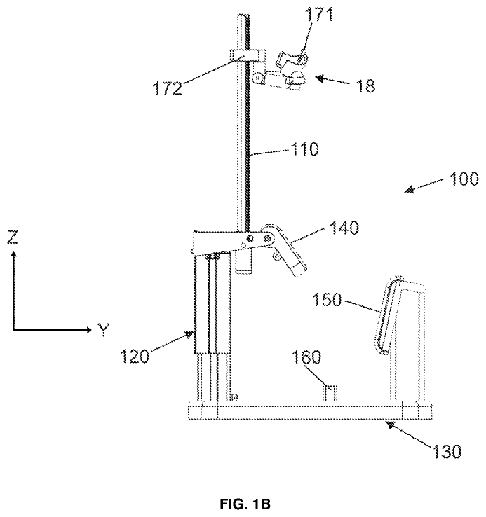

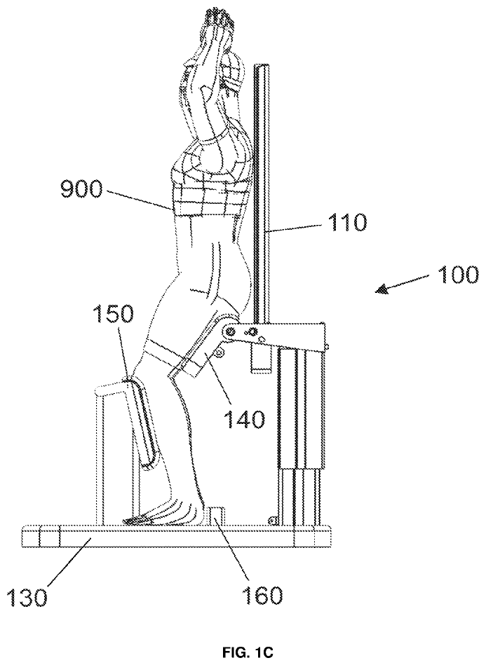

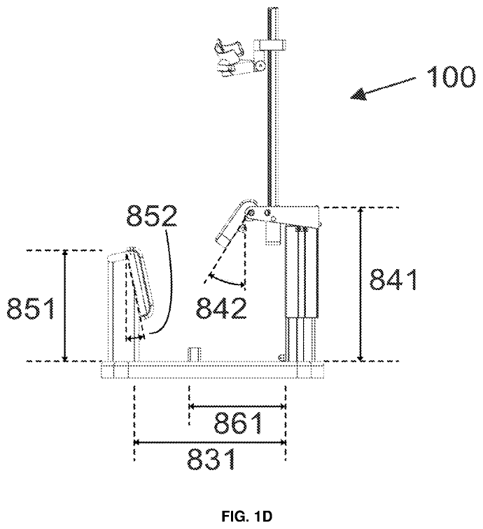

Embodiments of the technology described herein relate to translations along axes and/or rotations around axes. As shown in FIG. 1A and FIG. 1B, in some embodiments, a coordinate system is used that comprises an X axis, a Y axis, and a Z axis defined with respect to a patient support (e.g., a non integrated patient support and/or an integrated patient support) and/or a patient. In some embodiments, a coordinate system is used in which the X axis and Y axis together are in and/or define a horizontal plane and the Z axis is and/or defines a vertical axis. With respect to a patient positioned on a patient support (e.g., a patient positioning apparatus), the X axis is a left right, horizontal, or frontal axis; the Y axis is an anteroposterior, dorsoventral, or sagittal axis; and the Z axis is a sagittal or longitudinal axis. The X axis and the Y axis together are in and/or define a horizontal, transverse, and/or axial plane. The Y axis and the Z axis together are in and/or define a sagittal or longitudinal plane. The X axis and the Z axis together are in and/or define a frontal or coronal plane.

Accordingly, in some embodiments, descriptions of movements as “forward” or “backward” are movements along the Y axis; descriptions of movements as “left” or “right” are movements along the X axis; and descriptions of movements as “up” and “down” are movements along the Z axis. Furthermore, a rotation described as “roll” is rotation around the Y axis: a rotation described as “pitch” is rotation around the X axis; and a rotation described as “yaw” is rotation around the Z axis. Thus, in some embodiments, technologies are described as having six degrees of freedom, e.g., translations along one or more of the X, Y, and/or Z axes and rotations around one or more of the X, Y, and/or Z axes.

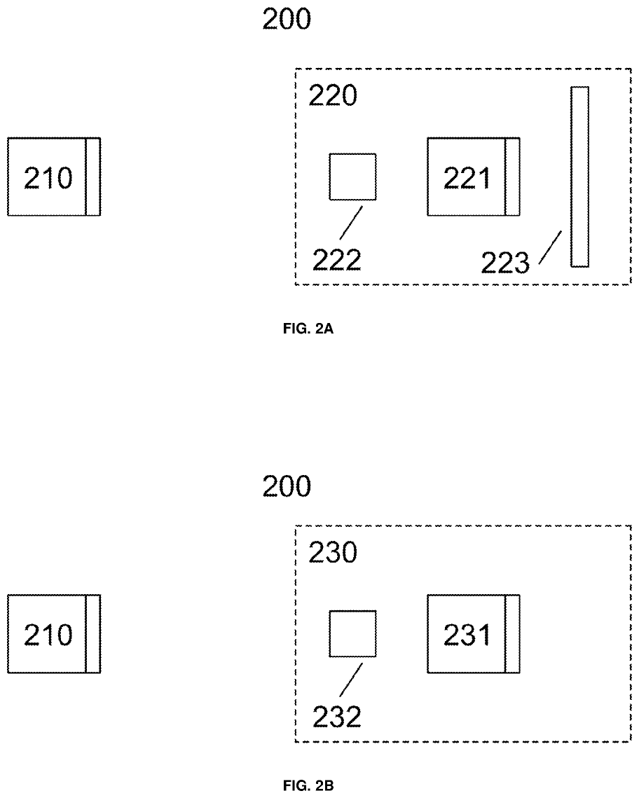

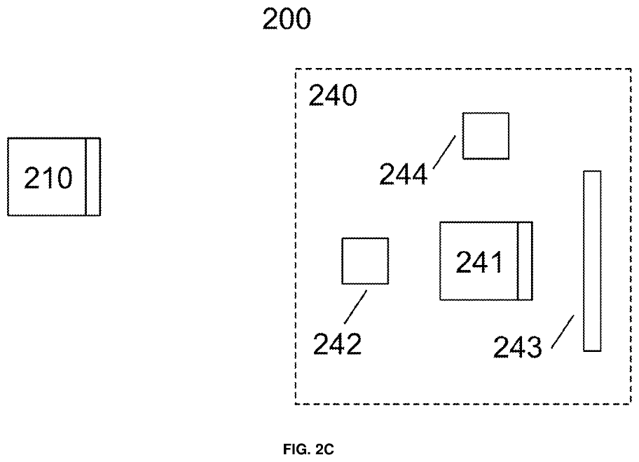

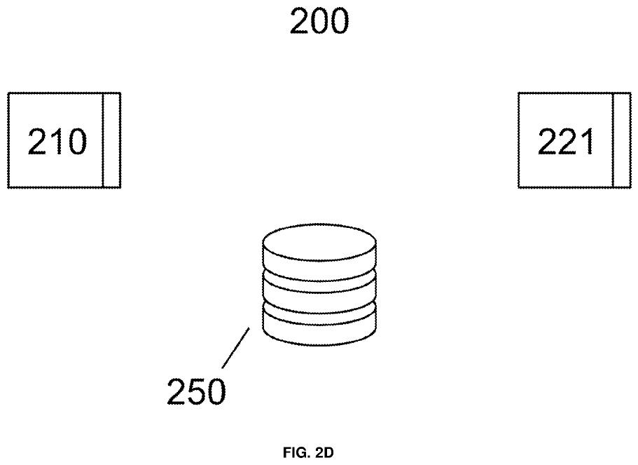

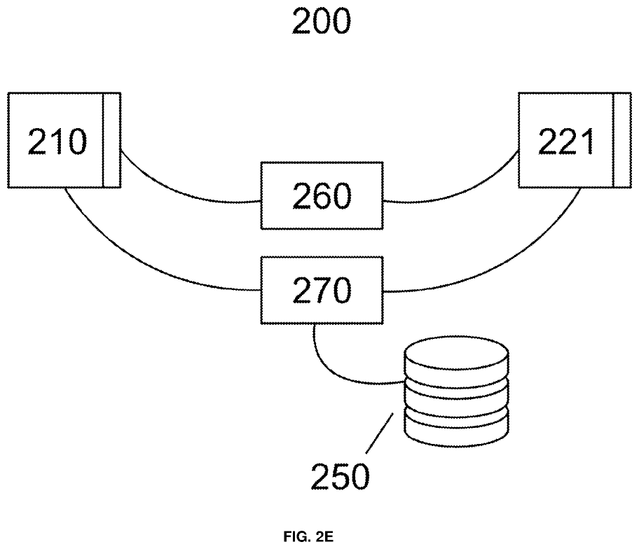

As used herein, the term “integrated patient support” refers to a patient support that is an integral component of a medical imaging system or of a medical therapy (e.g., radiotherapy) system. For example, medical imaging systems typically comprise a source and a detector arranged around an integrated patient support so that radiation from the source passes through a patient positioned on the integrated patient support and radiation passing through the patient is detected on the detector (see, e.g., feature 220 in FIG. 2A). A radiotherapy system typically comprises a source arranged around an integrated patient support so that radiation from the source contacts a region of the patient that is a target for radiotherapy (see, e.g., feature 230 in FIG. 2B). Medical imaging and radiotherapy systems often are installed in a radiation bunker to minimize radiation exposure to those outside the treatment area. Further, medical imaging and radiotherapy systems often comprise components (e.g., hardware and software) for control of the source, for data acquisition by the detector, and for data analysis and display.

As used herein, the term “non-integrated patient support” or “isolated patient support” refers to a patient support that is not integrated into a medical imaging or medical therapy (e.g., radiotherapy) system (see, e.g., feature 210 in FIG. 2A, FIG. 2B, and FIG. 2C). A non-integrated patient support is isolated from a medical imaging or medical therapy (e.g., radiotherapy) system and is thus provided apart from a medical imaging or medical therapy (e.g., radiotherapy) system. Typically, a non-integrated patient support is mounted on a support or a stand, or mounted to the floor, and the non integrated patient support may be provided in addition to an integrated patient support that is provided as an integral component of a medical imaging or medical therapy (e.g., radiotherapy) system.

As used herein, the term “patient support” may refer to an integrated patient support or to a non integrated patient support.

As used herein, the term “index” refers to a measurable position of a component of a patient support, e.g., an absolute position of the component in the coordinate space of the patient support (see FIG. 1A and FIG. 1B) or a relative position of a component defined by translation and/or rotation of the component of the patient support relative to another component of the patient support. In some embodiments, a component comprises a physical feature (e.g., a linear ruler or angular ruler) comprising a regular series of markings or other physical features (e.g., bumps, holes, stops, lines, dots, ridges, etc.) that provides the “index” for the component and where each marking or physical feature is associated with an “index value”. For example, a component may have thereon a series of markings providing an index for the component and where an individual marking (having an “index value”) is associated with a particular configuration of the component.

As used herein, the term “index value” refers to a particular value of an index that defines a configuration of a component of a patient support. An “index” may have a range of “index values” corresponding to the range of configurations available for the component. For example, a component that may be placed in a range of configurations over a distance of X units (e.g., cm) at increments of Y units (e.g., cm) would have X/Y index values, each of which defines one configuration of the component (e.g., with respect to the linear placement or position of the component). Similarly, a component that may be placed in a range of configurations through an angular range of X degrees at increments of Y degrees would have X/Y index values, each of which defines one configuration of the component (e.g., with respect to the angular placement or position of the component).

As used herein, the term “perched position” refers to a patient in a generally standing position with a torso angled posteriorly with respect to a vertical axis, optionally also having bent knees.

### Patient Support

In some embodiments, the technology relates to a non-integrated patient support. An exemplary patient support that finds use as a non-integrated patient support in the present technology is described (e.g., as a “patient support assembly” or, alternatively and interchangeably, as a “patient support”) in U.S. patent application Ser. No. 16/649,337 (published as U.S. Pat. App. Pub. No. 2020/0268327) or U.S. patent application Ser. No. 17/894,335, each of which is expressly incorporated herein by reference. As described herein, particular embodiments relate to a patient support (e.g., an integrated patient support and/or a non integrated patient support) that supports a patient in an upright or substantially upright position (e.g., standing, sitting, kneeling, perched). See, e.g., FIG. 1A and FIG. 1B. Imaging and/or treating patients in an upright position provides the benefits of increasing patient comfort. Further, imaging and/or treatment of patients in an upright position provides advantages over conventional diagnosis and/or treatment of patients in a horizontal position for many indications. As discussed herein, the technology provides a non-integrated patient support for determining a patient position (e.g., an upright position) and immobilization configuration that finds use in positioning the patient in the patient position (e.g., an upright position) on an integrated patient support for imaging and/or therapy.

For example, in some embodiments, the technology provides a non-integrated patient support as shown in FIG. 1A and FIG. 1B. In some embodiments (e.g., embodiments of systems discussed further hereinbelow), the technology relates to one or both of an integrated and/or a non integrated patient support. Accordingly, aspects of the technology that are common to both an integrated and a non-integrated patient support are discussed herein though particular embodiments of the technology relate to a nonconventional technology providing a non integrated patient support that is isolated from a medical imaging or medical therapy (e.g., radiotherapy) apparatus or system.

As shown in FIG. 1A and FIG. 1B, the patient support (e.g., integrated patient support or non integrated patient support) 100 comprises a back rest 110 for sustaining or supporting a back of the patient. The back rest 110 is coupled to a pillar or post 120 supporting the back rest 110. The pillar 120 is mounted to a platform, base, or surface 130 (e.g., a floor). In some embodiments, the patient support (e.g., integrated patient support or non integrated patient support) 100 is provided on a stand or other support (e.g., the pillar 120 is supported by a stand or support). In some embodiments, the patient support (e.g., integrated patient support or non-integrated patient support) 100 is mounted to a floor (e.g., the pillar 120 is mounted to a floor). In some embodiments, the platform 130 is mounted to a floor. In some embodiments, the patient support (e.g., integrated patient support or non integrated patient support) 100 comprises a leveling component (e.g., a spirit level, an accelerometer) to indicate the position and/or orientation of the non-integrated patient support relative to the Earth and/or a gravity vector, e.g., for use in installation, alignment, and adjustment of the non integrated patient support 100.

The patient support (e.g., integrated patient support or non integrated patient support) 100 further comprises a seat pan 140 for sustaining or supporting a posterior, buttocks, or thighs of the patient 900 (FIG. 1C). In some embodiments, the seat pan 140 is coupled to the pillar 120 in close proximity to the back rest 110. In some embodiments, the seat pan 140 is coupled to the back rest 110.

In some embodiments, the seat pan height 841, seat pan angle 842, shin rest height 851, shin rest angle 852, shin rest distance 831, and/or heel stop distance 861 are adjustable. FIG. 1D. In some embodiments, the back rest 110 and the seat pan 140 are rotatably coupled to the pillar 120 for adjusting the angle of the back rest 110 and/or of the seat pan 140 relative to the pillar 120 and/or relative to each other. In some embodiments, the seat pan 140 is rotatably coupled to the back rest 110 for adjusting the angle of the back seat pan 140 relative to the back rest 110.

Furthermore, embodiments provide that the vertical height of the pillar 120 is adjustable for adjusting the vertical height of the back rest 110 and seat pan 140.

Accordingly, in some embodiments, the pillar 120 is translatably coupled to a platform, base, stand, support, or surface 130 (e.g., a floor). In some embodiments, the seat pan 140 and/or the back rest 110 is/are translatably coupled to the pillar 120 for adjusting the height of the seat pan 140 and/or of the back rest 110 with respect to the pillar. In some embodiments, the seat pan 140 and/or the back rest 110 is/are rotatably coupled to the pillar 120 for adjusting the angle of the seat pan 140 and/or the back rest 110.

In some embodiments, the patient support 100 further comprises a shin rest 150 for sustaining or supporting shins of the patient 900 (e.g., a front part of the patient legs (e.g., between the knees and the ankles of the patient 900)). The shin rest 150 is coupled to the platform 130 in front of the back rest 110 and the seat pan 140. The shin rest 150 is offset from the back rest 110 such that it faces the shins of the patient when the patient 900 has her back positioned against the back rest 110. See FIG. 1C.

In some embodiments, the shin rest 150 is removably coupled to the platform 130 in front of the back rest 110 and the seat pan 140. For example, in some embodiments, the platform 130 comprises a plurality of holes 131 for receiving an apposite mating member of the shin rest 150 base. The holes 131 are provided at several horizontal offsets relative to the back rest 110, such that the horizontal distance between the back rest 110 and the shin rest 150 may be adjusted by placing the shin rest 150 in an appropriate hole 131 to provide a desired distance between the back rest 110 and the shin rest 150.

In some embodiments, other arrangements or mechanisms are provided for adjusting a horizontal distance between the shin rest 150 and the back rest 110. For example, in some embodiments, the shin rest 150 is translatably coupled to the platform 130. In some embodiments, the platform 130 comprises a rail and the shin rest 150 is translatably coupled to the rail. That is, in some embodiments, the platform 130 comprises a rail along which the shin rest 150 is moved (e.g., translated) and corresponding locking arrangements for fixing the shin rest 150 at a desired location. In another exemplary embodiment, the patient support (e.g., integrated patient support or non integrated patient support) comprises a motorized back rest (e.g., a back rest 110 operatively engaged with a back rest motor) and/or a motorized shin rest (e.g., a shin rest 150 operatively engaged with a shin rest motor), e.g., as described hereinbelow.

The back rest 110, the seat pan 140, and the shin rest 150 comprise substantially flat surfaces. In some embodiments, the back rest 110, the seat pan 140, and the shin rest 150 further comprise padding for accommodating a respective area of the body of the patient 900. FIG. 1C.

In some embodiments, the patient support 100 further comprises a pair of foot braces or a heel stop 160 for securing of the patient 900. See, e.g., FIG. 1C. In some embodiments, the foot braces are coupled to the platform 130 between the back rest 110 and the shin rest 150. The foot braces may comprise straps, clasps, or other arching pieces adapted to fasten over a top side of the patient feet for securing them to the platform 130. In some embodiments. the foot braces are coupled to the platform 130. In some embodiments, the foot braces are removably coupled to the platform 130. In some embodiments, the foot braces are translatably coupled to the platform 130. In some embodiments, the foot braces are adjustable by translating the foot braces. In some embodiments, the foot braces are translatably coupled to the platform 130 and may be locked into position. In some embodiments, the platform 130 comprises a plurality of holes for receiving an apposite mating member of the foot braces. The holes are provided at several horizontal offsets relative to the back rest 110, such that the horizontal distance between the back rest 110 and the foot braces may be adjusted by placing the foot braces in an appropriate hole to provide a desired distance between the back rest 110 and the foot braces. In some embodiments, the foot braces or heel stop 160 may be adjusted to be in a position that facilitates ingress or egress of patient 900 from the non-integrated patient support, e.g., by positioning the foot braces under the seat pan 140. In some embodiments, the patient support (e.g., integrated patient support or non-integrated patient support) 100 comprises motorized foot braces (e.g., foot braces operatively engaged with a foot brace motor).

In some embodiments, components other than foot braces are provided for immobilizing and/or stabilizing the patient 900, such as foot-shaped indentations, raised foot stops, or a heel stop 160. For example, in an exemplary embodiment, the patient support assembly 100 comprises a heel stop 160 coupled to the platform 130. In some embodiments, the heel stop 160 is removably coupled to the platform 130. In some embodiments, the heel stop 160 is translatably coupled to the platform 130. In some embodiments, the heel stop 160 comprises a padded member for receiving the heels of a patient 900. In some embodiments, the heel stop 160 is adjustable by translating the heel stop 160. In some embodiments, the heel stop 160 is translatably coupled to the platform 130 and may be locked into position. In some embodiments, the platform 130 comprises a plurality of holes for receiving an apposite mating member of the heel stop 160. The holes are provided at several horizontal offsets relative to the back rest 110, such that the horizontal distance between the back rest 110 and the heel stop 160 may be adjusted by placing the heel stop 160 in an appropriate hole to provide a desired distance between the back rest 110 and the heel stop 160. In some embodiments, the heel stop 160 may be adjusted to be in a position that facilitates ingress or egress of patient from the non integrated patient support, e.g., by positioning the heel stop 160 under the seat pan 140. In some embodiments, the patient support (e.g., integrated patient support or non integrated patient support) comprises a motorized heel stop (e.g., a heel stop 160 operatively engaged with a heel stop motor).

In some embodiments, the patient support (e.g., integrated patient support or non-integrated patient support) 100 further comprises arm rests or arm supports 170 for sustaining arms of the patient. Arm rests 170 are coupled to the back rest 110 at a left and right side of the back rest 110 to receive respective left and right arms of the patient. In some embodiments, the arm rests 170 are translatably coupled to the back rest 110. Accordingly, the vertical height of the arm rests 170, as well as their location relative to the back rest 110, may be adjusted (e.g., by translation) for accommodating patients of different sizes and different patient positions. Each arm rest 170 comprises a bent portion 171 for receiving a portion of the upper arm of the patient extending between the shoulder and the elbow. The bent portion 171 is rotatably coupled to arm 172, which is coupled to the back rest 110, for orienting the bent portion 171 to suit the patient physical dimensions. In some embodiments, the arm rests 170 sustain the arms of the patient in a predetermined position, such as an overhead position, or a downwards, lateral position such that the arms are positioned at respective left and right sides of the patient. In some embodiments, the patient support (e.g., integrated patient support or non-integrated patient support) comprises motorized arm rests (e.g., arm rests 170 operatively engaged with an arm rest motor).

As described herein, in some embodiments, the technology provides a patient support (e.g., integrated patient support or non integrated patient support) 100 that is configurable to support a patient in a position appropriate for medical imaging and/or treatment (e.g., radiotherapy). In some embodiments, the configurable patient support (e.g., integrated patient support or non-integrated patient support) 100 comprises one or more configurable and movable components, e.g., a back rest 110 (e.g., a configurable and movable back rest), an arm rest 170 (e.g., a configurable and movable arm rest), a seat pan 140 (e.g., a configurable and movable seat pan), a shin rest 150 (e.g., a configurable and movable shin rest), and/or a foot brace (e.g., a configurable and movable foot brace) or a heel stop 160 (e.g., a configurable and movable heel stop). In some embodiments, the patient support (e.g., integrated patient support or non-integrated patient support) further comprises a head rest (e.g., a configurable and movable head rest).

The patient support (e.g., integrated patient support or non-integrated patient support) 100 comprises components (e.g., back rest 110, arm rest 170, seat pan 140, shin rest 150, foot brace or heel stop 160, and head rest) that each may be placed in a number of positions to provide the patient support (e.g., integrated patient support or non-integrated patient support) in a particular configuration. In some embodiments, each component (e.g., back rest 110, arm rest 170, seat pan 140, shin rest 150. foot brace or heel stop 160, and head rest) is manipulable by a human user to place the component in the appropriate position for the desired configuration of the non-integrated patient support. Accordingly, embodiments provide that each component (e.g., back rest 110, arm rest 170, seat pan 140, shin rest 150, foot brace or heel stop 160, and head rest) may be moved (e.g., translation and/or rotated) by a human applying force to the component using her hands and no more than typical force provided by an average human. Embodiments provide that each component (e.g., back rest 110, arm rest 170, seat pan 140, shin rest 150, foot brace or heel stop 160, and head rest) may be locked into position (e.g., each component (e.g., back rest 110, arm rest 170, seat pan 140, shin rest 150, foot brace or heel stop 160, and head rest) may be placed in a locked state) to provide a static configuration of the patient support (e.g., integrated patient support or non integrated patient support) that stably supports a human in the appropriate position for imaging and/or treatment. Each component (e.g., back rest 110, arm rest 170, seat pan 140, shin rest 150, foot brace or heel stop 160, and head rest) may be unlocked (e.g., each component (e.g., back rest 110, arm rest 170, seat pan 140, shin rest 150, foot brace or heel stop 160, and head rest) may be placed in an unlocked state) so that one or more of the component(s) may be moved into a different position and thus provide a different configuration of the patient support (e.g., integrated patient support or non integrated patient support).

Each position of each component in a particular configuration of the patient support (e.g., integrated patient support or non-integrated patient support) may be described as having one or more of: a spatial position with respect to the coordinate system of the patient support (e.g., integrated patient support or non integrated patient support) (see FIG. 1A and FIG. 1B); a spatial position with respect to one or more other components; a rotation about an X, Y, and/or Z axis (e.g., roll, pitch, or yaw) of the coordinate system of the patient support (e.g., integrated patient support or non-integrated patient support) (see FIG. 1A and FIG. 1B); and/or an angle (“tilt” or “inclination”) with respect to one or more other components.

Thus, embodiments provide that each component (e.g., back rest 110, arm rest 170, seat pan 140, shin rest 150, foot brace or heel stop 160, and head rest) may be placed in a number of configurations that differ by increments of 1-cm to 5-cm translations in space (e.g., 1.0, 1.1, 1.2, 1.3, 1.4, 1.5, 1.6, 1.7, 1.8, 1.9, 2.0, 2.1, 2.2, 2.3, 2.4. 2.5, 2.6, 2.7, 2.8, 2.9, 3.0, 3.1, 3.2, 3.3, 3.4. 3.5, 3.6, 3.7, 3.8, 3.9, 4.0, 4.1, 4.2, 4.3, 4.4, 4.5. 4.6, 4.7, 4.8. 4.9, or 5.0 cm translations in space) and/or that differ by 1° to 5° rotations about an axis (e.g., 1.0, 1.1, 1.2, 1.3. 1.4, 1.5, 1.6, 1.7. 1.8, 1.9, 2.0. 2.1, 2.2, 2.3. 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0° rotations about an axis). Each of these configurations has an associated index value that unambiguously describes the position of the component; and, thus, a number of index values define the configuration of the patient support (e.g., integrated patient support or non-integrated patient support).

In particular, the back rest 110 may be configured to have a height (e.g., above the floor and/or above the platform or base 130) chosen from a plurality of heights that differ over a range from 1 to 300 cm at increments of 1 to 5 cm, e.g., to accommodate patients having a range of heights in a sitting or standing position. The back rest 110 may be configured to be positioned at angle with respect to the vertical Z axis (e.g., by rotation about the X axis) of the patient support (e.g., integrated patient support or non-integrated patient support) 100 (see FIG. 1A and FIG. 1B) chosen from a plurality of angles that differ over a range of approximately 1 to 50 degrees in increments of approximately 1 to 5 degrees, e.g., inclined anteriorly or posteriorly at an angle of from 0 to =25°(e.g., approximately +5°, ±10°, ±15°, ±20°, or =25°) with respect to the vertical Z axis.

Further, the seat pan 140 may be configured to have a height (e.g., above the floor and/or above the platform or base 130) chosen from a plurality of heights that differ over a range from 1 to 300 cm at increments of 1 to 5 cm, e.g., to accommodate patients having a range of heights in a sitting or standing position. In addition, the seat pan 140 may be configured to be positioned at an angle with respect to the horizontal Y axis (e.g., by rotation around the X axis) of the patient support (e.g., integrated patient support or non integrated patient support) (see FIG. 1A and FIG. 1B) chosen from a plurality of angles that differ over a range of approximately 1 to 40 degrees in increments of approximately 1 degree, e.g., inclined up or down at an angle of from 0 to ±20°(e.g., approximately +5°, ±10°, ±15°, or ±20°) with respect to the horizontal Y axis.

The shin rest 150 may be configured to have a distance from the back rest 110 and/or pillar 120 chosen from a plurality of distances that differ over a range from 1 to 50 cm at increments of 1 to 5 cm, e.g., to accommodate patients having a range of upper leg lengths. The shin rest 150 may also be configured to have a height (e.g., above the floor and/or above the platform or base 130) chosen from a plurality of heights that differ over a range from 1 to 50 cm at increments of 1 to 5 cm, e.g., to accommodate patients having a range of lower leg lengths. The shin rest 150 may be configured to be positioned at angle with respect to the vertical Z axis (e.g., by rotation about the X axis) of the patient support (e.g., integrated patient support or non integrated patient support) 100 (see FIG. 1A and FIG. 1B) chosen from a plurality of angles that differ over a range of approximately 1 to 30 degrees in increments of approximately 1 to 5 degrees, e.g., inclined at an angle of from 0 to 30°(e.g., approximately 5°, 10°, 15°, 20°, 25°, or 30°) with respect to the vertical Z axis. Further, in some embodiments, the shin rest 150 comprises two separate shin rest components (e.g., a left shin component and a right shin component) that each may be translated along the X axis over a range of approximately 1 to 20 cm (e.g., approximately 5, 10, 15, or 20 cm) to accommodate patients having a range of distances between legs and/or shins.

The foot brace or heel stop 160 may be configured to have a distance from the back rest 110 and/or pillar 120 chosen from a plurality of distances that differ over a range from 1 to 50 cm at increments of 1 to 5 cm, e.g., to accommodate patients having a range of leg lengths.

Each of the arm rests 170 may be configured to have a height (e.g., above the floor and/or above the platform or base 130) chosen from a plurality of heights that differ over a range from 1 to 150 cm at increments of 1 to 5 cm, e.g., to accommodate patients having a range of heights in a sitting or standing position, to accommodate patients having a range of arm lengths, and to maximize the number of independent configurations for each arm rest 170. Each arm 59 of each arm rest 170 and each bent portion 172 of each arm rest is independently rotatable through 180°around each of the X, Y, and Z axes to maximize the number of independent configurations for each arm rest 170.

The head rest may be configured to have a height (e.g., above the floor and/or above the platform or base 130) chosen from a plurality of heights that differ over a range from 1 to 50 cm at increments of 1 to 5 cm, e.g., to accommodate patients having a range of heights in a sitting or standing position.

Embodiments provide that each position of each component may be defined by an index that is associated with the component and a value for the index (“index value”) that unambiguously describes the position of the component. A set of index values may thus be used to unambiguously describe and define the configuration of the patient support (e.g., integrated patient support or non integrated patient support).

Thus, in some embodiments, the patient support (e.g., integrated patient support or non integrated patient support) 100 comprises markings and/or the patient support (e.g., integrated patient support or non-integrated patient support) 100 comprises components (e.g., back rest 110, arm rest 170, seat pan 140, shin rest 150, foot brace or heel stop 160, and head rest) comprising markings associated with the position of each of the components and the configuration of the patient support (e.g., integrated patient support or non integrated patient support). The markings may be used to observe and/or record the index value unambiguously describing and unambiguously defining the position of each component.

In some embodiments, each component (e.g., back rest 110, arm rest 170, seat pan 140, shin rest 150, foot brace or heel stop 160, and head rest) comprises a sensor that outputs a signal describing the value of the component index describing the position of the component. In some embodiments, a switch is associated with each indexed position of each component and the “on” or “off” state of each switch indicates the position of each component and thus indicates the index value for the position of the component. Accordingly, in some embodiments, index values for each component are encoded in an electronic (e.g., analog or digital) signal. In some embodiments, component identifying information (e.g., a component name) and/or a number of component index values are provided on a display for observation by a user and, in some embodiments, recording by a user. In some embodiments, component identifying information (e.g., component name) and/or a number of component index values are provided on a number of dials, a light emitting diode (LED) display, a liquid crystal display (LCD). or other visual representation that may be observed by a user.

In some embodiments, the index values are recorded on a tangible storage medium (e.g., random access memory, a hard drive, flash memory, or any other machine readable storage medium). In some embodiments, the index values are recorded in a database that is stored on a tangible storage medium. In some embodiments, the index values are associated with an identifier of a particular patient for which the index values define a configuration of a patient support appropriate for the imaging and/or treatment of the particular patient. In some embodiments, the index values are associated with demographic information (e.g., patient height, weight, biological sex, race or ancestry, body type) for which the index values define a configuration of a patient support appropriate for the imaging and/or treatment of a member of the demographic group. Embodiments provide that the index values may be recalled from a tangible storage medium for use in configuring a patient support for imaging and/or treatment of a patient.

Accordingly, embodiments relate to a data structure comprising one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value. In some embodiments, the data structure further comprises patient information (e.g., unique patient identifier, patient first name, patient last name, patient medical condition, demographic information (e.g., patient height, weight, biological sex, race or ancestry, body type, etc.), etc.) In some embodiments, the data structure further comprises demographic information for a patient class (e.g., height. weight, biological sex, race or ancestry, body type, etc.)

In some embodiments, the index values associated with a demographic class provide a configuration preset for a patient support, e.g., to provide a starting point configuration that is modified for a particular patient to provide a specific configuration for the patient. Accordingly, index values associated with demographic groups improve the efficiency of configuring a patient support by decreasing the time associated with adjusting each component for the particular patient and patient position appropriate for imaging and/or therapy of the patient.

### Motorized Patient Support

In some embodiments, one or more configurable and movable components of the patient support (e.g., integrated patient support or non-integrated patient support) 100 comprises one or more motorized components, e.g., a motorized back rest (e.g., a back rest 110 operatively engaged with a back rest motor), a motorized head rest (e.g., a head rest operatively engaged with a head rest motor), a motorized arm rest (e.g., an arm rest 170 operatively engaged with an arm rest motor), a motorized seat pan (e.g., a seat pan 140 operatively engaged with a seat pan motor), a motorized shin rest (e.g., a shin rest 150 operatively engaged with a shin rest motor), and/or a motorized foot brace (e.g., a foot brace operatively engaged with a foot brace motor). In some embodiments, the motorized foot brace is a motorized heel stop (e.g., a heel stop 160 operatively engaged with a heel stop motor).

In some embodiments, the back rest motor is structured to move (e.g., translate and/or rotate) the back rest 110, the head rest motor is structured to move (e.g., translate and/or rotate) the head rest, the arm rest motor is structured to move (e.g., translate and/or rotate) the arm rest 170, the seat pan motor is structured to move (e.g., translate and/or rotate) the seam member 140, the shin rest motor is structured to move (e.g., translate and/or rotate) the shin rest 150, and/or the foot brace motor or heel stop motor is structured to move (e.g., translate and/or rotate) the foot brace or the heel stop. In some embodiments, the technology provides a component (e.g., a computer, a microcontroller, and/or a microprocessor) configured to coordinate control and/or movement of one or more of the motorized components, e.g., the motorized back rest, the motorized head rest, the motorized arm rest, the motorized seat pan, the motorized shin rest, and/or the motorized foot brace or motorized heel stop, to provide the patient support (e.g., integrated patient support or non integrated patient support) into one or more specific configurations comprising the motorized back rest, the motorized head rest, the motorized arm rest, the motorized seat pan, the motorized shin rest, and/or the motorized foot brace or motorized heel stop in specified positions.

In some embodiments, the component (e.g., the computer, the microcontroller, and/or the microprocessor) configured to coordinate control and/or movement of the motorized components (e.g., the motorized back rest, the motorized head rest, the motorized arm rest, the motorized seat pan, the motorized shin rest, and/or the motorized foot brace or motorized heel stop) to provide the patient support (e.g., integrated patient support or non integrated patient support) 100 into one or more specific configurations is configured to actuate and/or control the back rest motor, the head rest motor, the arm rest motor, the seat pan motor, the shin rest motor, and/or the foot brace motor or heel stop motor. In some embodiments, actuating one or more of the motorized components (e.g., the motorized back rest, the motorized head rest, the motorized arm rest, the motorized seat pan, the motorized shin rest, and/or the motorized foot brace or motorized heel stop) comprises setting one or more of the motorized components into an “on” state (e.g., by providing electric current and/or electric voltage to the one or more motorized components (e.g., to a motor operatively coupled to a motorized component)) or into an “off” state (e.g., by eliminating electric current and/or electric voltage to the one or more motorized components (e.g., to a motor operatively coupled to a motorized component)). In some embodiments, actuating one or more of the motorized components (e.g., the motorized back rest, the motorized head rest, the motorized arm rest, the motorized seat pan, the motorized shin rest, and/or the motorized foot brace or motorized heel stop) comprises controlling the linear and/or rotational speed: and/or controlling the linear and/or rotational acceleration of the one or more motorized components (e.g., by controlling the linear and/or rotational speed:

and/or controlling the linear and/or rotational acceleration of a motor operatively coupled to a motorized component).

In some embodiments, the technology comprises software (e.g., a software object) comprising instructions for a specified position and/or specified movement (e.g., coordinated movement) of one or more of the motorized components, e.g., the motorized back rest, the motorized head rest, the motorized arm rest, the motorized seat pan, the motorized shin rest, and/or the motorized foot brace or motorized heel stop; and associated computer memory to store the software and/or to store data describing one or more positions of the configurable patient support and/or one or more positions of the motorized components of the configurable patient support. In some embodiments, a method to move one or more of the motorized components is provided as an object method.

In some embodiments, data and/or a data structure describing one or more positions and/or one or more specified movements of the motorized components is provided as an object data structure. Some embodiments provide an object oriented pipeline for moving one or more of the motorized components, e.g., comprising one or more software objects, to move one or more of the motorized components.

In some embodiments, a position of the patient support (e. g., integrated patient support or non integrated patient support) 100 and/or a position of the motorized components of the configurable patient support is specific for an individual patient; is specific for an individual imaging and/or treatment plan; and/or is specific for an individual imaging and/or treatment plan for an individual patient. In some embodiments, data and/or a data structure describing a position of the motorized components of the configurable patient support is provided as an object data structure. As described above, embodiments provide that each position of each component may be defined by an index that is associated with the component and a value for the index (“index value”) that unambiguously describes the position of the component. A set of index values may thus be used to unambiguously describe and define the configuration of the patient support (e.g., integrated patient support or non-integrated patient support) and, accordingly, be provided as inputs into software that provides instructions to a microprocessor and motors for positioning components (e.g., motorized back rest, motorized arm rest, motorized seat pan, motorized shin rest, motorized foot brace or heel stop, and/or motorized head rest) of the patient support (e.g., integrated patient support or non integrated patient support) 100 to provide a defined configuration of the patient support (e.g., integrated patient support or non-integrated patient support) 100.

In some embodiments, each component (e.g., motorized back rest, motorized arm rest, motorized seat pan, motorized shin rest, motorized foot brace or heel stop, and/or motorized head rest) comprises a sensor that outputs a signal describing the component, the component index, and/or the value of the component index describing the position of the component. In some embodiments, a switch is associated with each indexed position of each component and the “on” or “off” state of each switch indicates the position of each component and thus indicates the index value for the position of the component.

Accordingly, in some embodiments, index values for each component are encoded in an electronic (e.g., analog or digital) signal. In some embodiments, component identifying information (e.g., a component name) and/or a number of component index values are provided on a display for observation by a user and, in some embodiments, recording by a user. In some embodiments, component identifying information (e.g., a component name) and/or a number of component index values are provided on a number of dials, a light emitting diode (LED) display, a liquid crystal display (LCD), or other visual representation that may be observed by a user.

In some embodiments, the index values are recorded on a tangible storage medium (e.g., random access memory, a hard drive, flash memory, or any other machine readable storage medium). In some embodiments, the index values are recorded in a database that is stored on a tangible storage medium. In some embodiments, the index values are associated with an identifier of a particular patient for which the index values define a configuration of a patient support appropriate for the imaging and/or treatment of the particular patient. In some embodiments, the index values are associated with demographic information (e.g., height, weight, biological sex. race or ancestry, body type) for which the index values define a configuration of a patient support appropriate for the imaging and/or treatment of a member of the demographic group. Embodiments provide that the index values may be recalled from a tangible storage medium for use in configuring a patient support for imaging and/or treatment of a patient.

Accordingly, embodiments relate to a data structure comprising one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value. In some embodiments, the data structure further comprises patient information (e.g., unique patient identifier, patient first name, patient last name, patient medical condition, demographic information (e.g., patient height, weight, biological sex, race or ancestry, body type, etc.), etc.) In some embodiments, the data structure further comprises demographic information for a patient class (e.g., height, weight, biological sex, race or ancestry, body type).

In some embodiments, the index values associated with a demographic class provide a configuration preset for a patient support, e.g., to provide a starting point configuration that is modified for a particular patient to provide a specific configuration for the patient. Accordingly, index values associated with demographic groups improve the efficiency of configuring a patient support by decreasing the time associated with adjusting each component for the particular patient and patient position appropriate for imaging and/or therapy of the patient.

### Systems

In some embodiments, e.g., as shown in FIG. 2A, FIG. 2B, and FIG. 2C, the technology provides a system comprising a non integrated patient support and an integrated patient support. As shown in FIG. 2A, FIG. 2B, and FIG. 2C, embodiments of the system 200 comprise a non integrated patient support 210 and an integrated patient support 221, or 231, or 241. In some embodiments, a system 200 comprises a non integrated patient support 210 and a medical imaging subsystem 220 comprising a source 222, a detector 223, and an integrated patient support 221 (FIG. 2A). In some embodiments, a system 200 comprises a non integrated patient support 210 and a radiotherapy subsystem 230 comprising a source 232 and an integrated patient support 231 (FIG. 2B). In some embodiments, a system 200 comprises a non integrated patient support 210; and a medical imaging and radiotherapy subsystem 240 comprising a first (e.g., imaging) source 242 and a detector 243 for medical imaging, a second (e.g., therapy) source 244 for radiotherapy, and an integrated patient support 241 used for both medical imaging and radiotherapy (FIG. 2C).

In some embodiments, a system 200 comprises a non integrated patient support 210, an integrated patient support 221, and a database 250 (see, e.g., FIG. 2D). In some embodiments, the database 250 is stored on a tangible storage medium. In some embodiments, the database 250 is stored on a tangible storage medium locally or in the cloud. In some embodiments, the database 250 is stored as a written record.

In some embodiments, the system 200 comprises a non-integrated patient support 210, an integrated patient support 221, a database 250, a wireless and/or a wired communications medium for communicating data between the non integrated patient support 210 and the database 250, and a wireless and/or a wired communications medium for communicating data between the integrated patient support 210 and the database 250 (FIG. 2E).

While embodiments comprising a database 250 (and, optionally, a power supply 260 and/or a computer (e.g., a microprocessor) 270) and an integrated patient support 221 are discussed herein and are shown in FIG. 2D and FIG. 2E, the technology includes similar embodiments related to a system comprising a non-integrated patient support, an integrated patient support 231 provided as a component of a radiotherapy subsystem 230 (FIG. 2B), and a database (and, optionally, a power supply and/or a computer (e.g., a microprocessor)); and to a system comprising a non-integrated patient support, an integrated patient support 241 provided as a component of a medical imaging and radiotherapy subsystem 240 (FIG. 2C), and a database (and, optionally, a power supply and/or a computer (e.g., a microprocessor)). Accordingly, a description of embodiments comprising a database (and, optionally, a power supply and/or a computer (e.g., a microprocessor) as described further below) that refers to an integrated patient support 221 is intended to refer to an integrated patient support 221, 231, or 241 as discussed herein.

In some embodiments, the database 250 comprises information describing a configuration of a patient support (e.g., a non integrated patient support 210 and/or an integrated patient support 221). In some embodiments, the information describing a configuration of a patient support is recorded after configuring the non-integrated patient support 210 and positioning a patient on the non-integrated patient support 210 in a position appropriate for imaging and/or treatment. In some embodiments, the database 250 comprises data describing one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value. In some embodiments, the database 250 comprises data describing a patient (e.g., unique patient identifier, patient first name, patient last name, patient medical condition, demographic information (e.g., height, weight, biological sex, race or ancestry, body type, etc.), etc.) that is associated with a particular configuration for use with the patient and described by one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value.

In some embodiments, the database 250 comprises data describing a patient class, e.g., data describing demographic information for a patient class (e.g., height, weight, biological sex, race or ancestry, body type), that is associated with a particular configuration for use with the patient class and described by one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value.

In some embodiments, data stored in database 250 (e.g., one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value) are used to configure an integrated patient support 221 using the one or more of a back rest height index value. a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value.

In some embodiments, patient support configuration data stored in database 250 (one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value) are used to configure an integrated patient support 221 using the one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value for a patient identified by patient data stored in the database 250 (e.g., unique patient identifier, patient first name, patient last name, patient medical condition, demographic information (e.g., height, weight, biological sex, race or ancestry, body type, etc.), etc.) and associated with the patient support configuration data.

In some embodiments, patient support configuration data stored in database 250 (one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value) are used to configure an integrated patient support 221 using the one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value for a patient class identified by patient class data stored in the database 250 (e.g., height, weight, biological sex, race or ancestry, body type) and associated with the patient support configuration data.

In some embodiments, a system comprises a motorized patient support as described herein, e.g., a patient support (e.g., an integrated patient support and/or a non integrated patient support) comprising one or more motorized components, e.g., a motorized back rest (e.g., a back rest operatively engaged with a back rest motor), a motorized head rest (e.g., a head rest operatively engaged with a head rest motor), a motorized arm rest (e.g., an arm rest operatively engaged with an arm rest motor), a motorized seat pan (e.g., a seat pan operatively engaged with a seat pan motor), a motorized shin rest (e.g., a shin rest operatively engaged with a shin rest motor), and/or a motorized foot brace (e.g., a foot brace operatively engaged with a foot brace motor) or a motorized heel stop (e.g., a heel stop operatively engaged with a heel stop motor).

In some embodiments, a system comprises a motorized patient support as described herein, e.g., a patient support (e.g., an integrated patient support and/or a non integrated patient support) comprising one or more motorized components, e.g., a motorized back rest (e.g., a back rest operatively engaged with a back rest motor), a motorized head rest (e.g., a head rest operatively engaged with a head rest motor), a motorized arm rest (e.g., an arm rest operatively engaged with an arm rest motor), a motorized seat pan (e.g., a seat pan operatively engaged with a seat pan motor), a motorized shin rest (e.g., a shin rest operatively engaged with a shin rest motor), and/or a motorized foot brace (e.g., a foot brace operatively engaged with a foot brace motor) or a motorized heel stop (e.g., a heel stop operatively engaged with a heel stop motor); and a power supply structured to provide power to one or more of a back rest motor, a head rest motor, an arm rest motor, a seat pan motor, a shin rest motor, and/or a foot brace motor or a heel stop motor.

In some embodiments, e.g., as shown in FIG. 2E, a system 200 comprises a motorized non integrated patient support 210 (e.g., a non integrated patient support comprising one or more motorized components, e.g., a motorized back rest (e.g., a back rest operatively engaged with a back rest motor), a motorized head rest (e.g., a head rest operatively engaged with a head rest motor), a motorized arm rest (e.g., an arm rest operatively engaged with an arm rest motor), a motorized seat pan (e.g., a seat pan operatively engaged with a seat pan motor), a motorized shin rest (e.g., a shin rest operatively engaged with a shin rest motor), and/or a motorized foot brace (e.g., a foot brace operatively engaged with a foot brace motor) or a motorized heel stop (e.g., a heel stop operatively engaged with a heel stop motor)); a motorized integrated patient support 221 (e.g., an integrated patient support comprising one or more motorized components, e.g., a motorized back rest (e.g., a back rest operatively engaged with a back rest motor), a motorized head rest (e.g., a head rest operatively engaged with a head rest motor), a motorized arm rest (e.g., an arm rest operatively engaged with an arm rest motor), a motorized seat pan (e.g., a seat pan operatively engaged with a seat 35 pan motor), a motorized shin rest (e.g., a shin rest operatively engaged with a shin rest motor). and/or a motorized foot brace (e.g., a foot brace operatively engaged with a foot brace motor) or a motorized heel stop (e.g., a heel stop operatively engaged with a heel stop motor)): a power supply 260 structured to provide power to one or more of a back rest motor, a head rest motor, an arm rest motor, a seat pan motor, a shin rest motor, and/or a foot brace motor or a heel stop motor of the motorized non integrated patient support 210 and/or of the motorized integrated patient support 221; a computer (e.g., a microprocessor) 270, and a database 250. In some embodiments, the computer 270 controls one or more motorized components of the integrated patient support 221 and/or the non-integrated patient support 210 according to software instructions recorded on a tangible medium to configure the integrated patient support 221 and/or the non-integrated patient support 210 according to patient support configuration data (one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value), e.g., patient support configuration data stored in the database 250. In some embodiments, the computer 270 records (e.g., in the database 250) one or more index value (one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value) describing the configuration of the motorized non-integrated patient support 210 and/or of the motorized integrated patient support 221.

In some embodiments, systems comprise a computer and/or data storage provided virtually (e.g., as a cloud computing resource). In some embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

In some embodiments, one or more components are provided in individual software objects connected in a modular system. In some embodiments, the software objects are extensible and portable. In some embodiments, the objects comprise data structures and operations that transform the object data. In some embodiments, the objects are used by manipulating their data and invoking their methods. Accordingly, embodiments provide software objects that imitate, model, or provide concrete entities, e.g., for numbers, shapes, data structures, that are manipulable. In some embodiments, software objects are operational in a computer or in a microprocessor. In some embodiments, software objects are stored on a computer readable medium.

Embodiments comprise use of code that produces and manipulates software objects, e.g., as encoded using a language such as but not limited to BASIC, Java, C. C++, C#, Python, PHP, Ruby, Perl, MATLAB, Mathematica, Object Pascal, Objective-C, Swift, Scala, Common Lisp, and Smalltalk.

### Installation Leveling Adjustment

In some embodiments, a plurality of adjusters finds use in levelling a patient support (e.g., an integrated patient support and/or a non integrated patient support), e.g., during installation or from time to time to adjust the position of the patient support. In some embodiments, the adjusters are as described in U.S. patent application Ser. No. 17/894,335, which is incorporated herein by reference. The adjustors each have an X. Y, and Z adjustment for leveling. For example, the adjustor can be adjusted in the Z-direction and then radially adjusted to provide an X and/or Y direction adjustment. In some embodiments, the X and Y adjustment is independent from (does not affect) the Z adjustment.

During installation, the adjustors are positioned to define a level plane in a vertical direction (e.g., Z-direction). Then, one adjustor is moved to achieve the desired position in a first direction (e.g., X direction) and the other two adjustors are moved to achieve the desired position in the second direction (e.g., Y-direction). In some embodiments, the adjustors provide an adjustment range of approximately ±2.5 mm (e.g., approximately 2 to 3 mm (e.g., 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00 mm)).

### Vac-Bag

In some embodiments, a vac-bag finds use on the patient support described herein (e.g., an integrated patient support and/or a non integrated patient support). In some embodiments, the vac-bag is as described in U.S. patent application Ser. No. 17/894,335, which is incorporated herein by reference. For example, in some embodiments, vac-bags are provided on the seat pan, backrest, and/or arm rests. The vac-bag is a bag containing “beans” (e.g., polymer (e.g., expanded polystyrene or the like) spheres as in a bean bag) and is squishy and malleable prior to evacuation. Anatomy is pressed into the vac-bag and the vac-bag is evacuated to lock the beans into place (e.g., mold the vac-bag to a patient to provide a custom fit contour) to provide a patient customized vac-bag. The patient customized vac-bag can then be removed, stored, and utilized again at a later time for the patient. In some embodiments, the vac-bag comprises a number of interior sections to minimize movement of the beans, e.g., as described in U.S. patent application Ser. No. 17/894,335, which is incorporated herein by reference.

In some embodiments, the technology provides a method of conforming a vac-bag to a patient. Methods comprise providing a non integrated patient support and providing a vac-bag on the seat pan, back rest, and/or arm rests of the non integrated patient support to provide a non integrated patient support comprising a vac-bag.

Further, methods comprise positioning a patient on the non integrated patient support comprising a vac bag and evacuating a portion of the air within the vac-bag to provide a contoured vac-bag specific for the shape of the patient. In some embodiments, methods comprise storing the contoured vac-bag for use at a later time for the patient. In some embodiments, methods comprise providing an integrated patient support and providing the contoured vac-bag on the seat pan, back rest, and/or arm rests of the integrated patient support to provide an integrated patient support comprising a contoured vac-bag. In some embodiments, methods comprise positioning the patient on the integrated patient support comprising the contoured vac-bag. In some embodiments, vac-bags and the seat pan, back rest, and/or arm rests of the patient support (e.g., non integrated patient support and/or integrated patient support) comprise complementary (e.g., mated) components of a keyed interface (e.g., key and groove) to maximize correct positioning of the vac-bag on the integrated patient support. For example, in some embodiments, the surface of the vac-bag that contacts the seat pan, back rest, and/or arm rests comprises a key that is complementary to and positioned within a corresponding notch provided on the seat pan, back rest, and/or arm rests of the non-integrated patient support and the integrated patient support.

### Methods

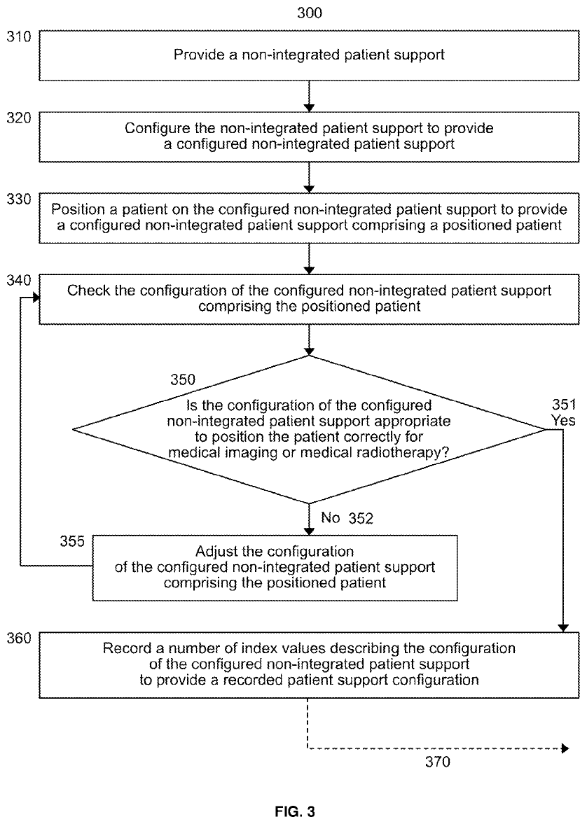

In some embodiments, the technology provides methods related to the non integrated patient support and systems comprising the non integrated patient support described herein. For instance, as shown in FIG. 3, embodiments of methods 300 comprise providing 310 a non-integrated patient support as described herein: and configuring 320 the non-integrated patient support (e.g., by positioning one or more configurable and movable components of the non integrated patient support (e.g., a back rest, an arm rest, a seat pan, a shin rest, and/or a foot brace or heel stop)) to provide a configured non integrated patient support. In some embodiments, positioning one or more configurable and movable components of the non integrated patient support comprises manually translating and/or rotating one or more configurable and movable components of the non integrated patient support (e.g., a back rest, an arm rest, a seat pan, a shin rest, and/or a foot brace or heel stop). In some embodiments, positioning one or more configurable and movable components of the non integrated patient support comprises translating and/or rotating one or more configurable and movable components of the non-integrated patient support (e.g., a back rest, an arm rest, a seat pan, a shin rest, and/or a foot brace or heel stop) by activating one or more motorized components, e.g., a motorized back rest (e.g., a back rest operatively engaged with a back rest motor), a motorized head rest (e.g., a head rest operatively engaged with a head rest motor), a motorized arm rest (e.g., an arm rest operatively engaged with an arm rest motor), a motorized seat pan (e.g., a seat pan operatively engaged with a seat pan motor), a motorized shin rest (e.g., a shin rest operatively engaged with a shin rest motor), and/or a motorized foot brace (e.g., a foot brace operatively engaged with a foot brace motor or a motorized heel stop (e.g., a heel stop operatively engaged with a heel stop motor).

Next, the method 300 comprises positioning 330 a patient on the configured non-integrated patient support to provide a configured non integrated patient support comprising a positioned patient; checking 340 the configuration of the configured non-integrated patient support comprising the positioned patient; and deciding 350 if the configuration of the configured non integrated patient support comprising the patient is appropriate to position the patient correctly for medical imaging and/or radiotherapy (351) or if the configuration of the configured non integrated patient support comprising the patient is not appropriate to position the patient correctly for medical imaging and/or radiotherapy (352). In some embodiments, checking 340 the configuration of the configured non-integrated patient support comprising the positioned patient comprises a clinician visually checking the configuration. In some embodiments, checking 340 the configuration of the configured non integrated patient support comprising the positioned patient comprises comparing an image of the configured non integrated patient support with a reference image of a patient support in a correct configuration. In some embodiments, checking 340 the configuration of the configured non integrated patient support comprising the positioned patient comprises comparing one or more index values for a number of components of the patient support with one or more reference index values of a patient support in a correct configuration. In some embodiments, checking 340 the configuration of the configured non-integrated patient support comprising the positioned patient comprises simulating medical imaging (e.g., simulating a CT scan) or simulating radiotherapy of the patient.

If the deciding step 350 identifies the configured non integrated patient support as not being appropriate to position the patient correctly for medical imaging and/or radiotherapy (352), then the method 300 comprises adjusting 355 the configuration of the configured non integrated patient support comprising the positioned patient: and repeating the steps of checking 340 the configuration of the configured non-integrated patient support comprising the positioned patient; and deciding 350 if the configuration of the configured non integrated patient support comprising the patient is appropriate to position the patient correctly for medical imaging and/or radiotherapy (351) or if the configuration of the configured non integrated patient support comprising the patient is not appropriate to position the patient correctly for medical imaging and/or radiotherapy (352). After performing a number of checking 340 and deciding 350 steps, and performing a number of adjusting 355 step(s) if needed, to identify or verify that the configuration of the configured non integrated patient support comprising the patient is appropriate to position the patient correctly for medical imaging and/or radiotherapy (351), then the method 300 comprises recording 360 a number of index values describing the configuration of the non integrated patient support to provide a recorded patient support configuration (e.g., comprising one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value). In some embodiments, recording 360 a number of index values describing the configuration of the non-integrated patient support comprises manually recording the number of index values. In some embodiments, recording 360 a number of index values describing the configuration of the non integrated patient support comprises storing the number of index values in a database.

In some embodiments, checking and deciding steps, and adjusting step(s) if needed, are performed after configuring 320 the non integrated patient support and prior to positioning the patient on the configured non integrated patient support.

In some embodiments, a method 300 comprises using 370 a number of index values describing the configuration of the non-integrated patient support in a method for configuring an integrated patient support (e.g., according to an embodiment of method 400).

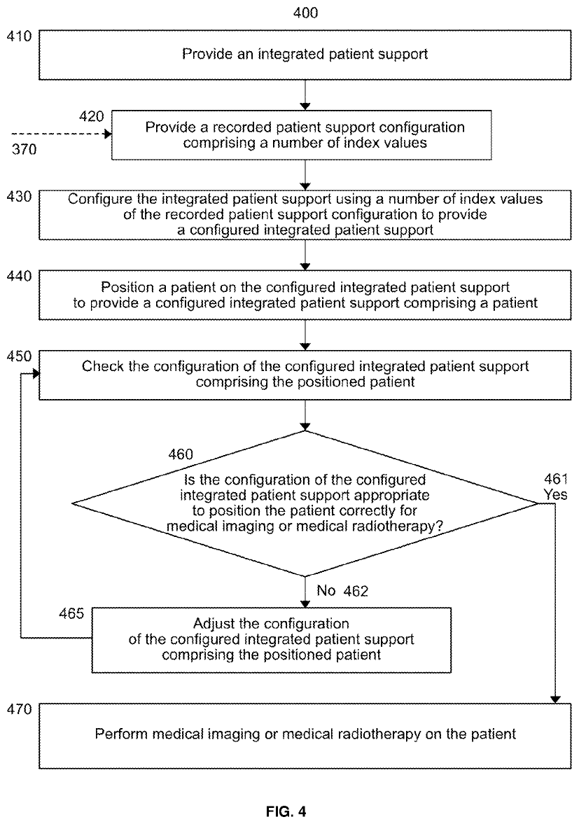

In some embodiments, the technology provides methods related to configuring an integrated patient support and systems comprising a non-integrated and an integrated patient support as described herein. For instance, as shown in FIG. 4, embodiments of methods 400 comprise providing 410 an integrated patient support (e.g., a patient support that is an integral component of a medical imaging or medical radiotherapy system); and providing 420 a recorded patient support configuration comprising a number of index values describing a configuration of an integrated patient support (e.g., comprising one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value). In some embodiments, the index values describing the configuration of the integrated patient support are provided by recording 360 a number of index values describing the configuration of the non-integrated patient support in an embodiment of a method 300. For instance, in some embodiments, a number of index values are retrieved from a database comprising recorded index values provided by an embodiment of method 300.

In some embodiments, methods 400 comprise identifying a patient and providing 420 a recorded patient support configuration by retrieving recorded index values provided by an embodiment of method 300 previously performed for the patient. In some embodiments, providing 420 a recorded patient support configuration comprises identifying a patient by acquiring patient identification information (e.g., a unique patient identifier, patient first name, patient last name, patient medical condition, and/or demographic information (e.g., height, weight, biological sex, race or ancestry, body type, etc.), etc.)) of the patient and comparing the patient identification information with stored patient information (e.g., a unique patient identifier, patient first name, patient last name, patient medical condition, and/or demographic information (e.g., height, weight, biological sex, race, body type, etc.), etc.)) stored in a database and associated with a number of stored index values describing the configuration of the integrated patient support to be used with the patient.

In some embodiments, methods 400 comprise configuring 430 the integrated patient support by positioning one or more configurable and movable components of the integrated patient support (e.g., a back rest, an arm rest, a seat pan, a shin rest, and/or a foot brace or heel stop)) according to the recorded patient support configuration comprising a number of index values (e.g., comprising one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value) to provide a configured integrated patient support.

In some embodiments, positioning one or more configurable and movable components of the integrated patient support comprises manually translating and/or rotating one or more configurable and movable components of the non integrated patient support (e.g., a back rest, an arm rest, a seat pan, a shin rest, and/or a foot brace or heel stop) to place each component according to the recorded patient support configuration (e.g., according to one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value).

In some embodiments, positioning one or more configurable and movable components of the integrated patient support comprises translating and/or rotating one or more configurable and movable components of the integrated patient support (e.g., a back rest, an arm rest, a seat pan, a shin rest, and/or a foot brace or heel stop) by activating one or more motorized components, e.g., a motorized back rest (e.g., a back rest operatively engaged with a back rest motor), a motorized head rest (e.g., a head rest operatively engaged with a head rest motor), a motorized arm rest (e.g., an arm rest operatively engaged with an arm rest motor), a motorized seat pan (e.g., a seat pan operatively engaged with a seat pan motor), a motorized shin rest (e.g., a shin rest operatively engaged with a shin rest motor), and/or a motorized foot brace (e.g., a foot brace operatively engaged with a foot brace motor or a motorized heel stop (e.g., a heel stop operatively engaged with a heel stop motor) to place each component according to the recorded patient support configuration (e.g., according to one or more of a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value).

Next, the method 400 comprises positioning 440 a patient on the configured integrated patient support to provide a configured integrated patient support comprising a positioned patient; checking 450 the configuration of the configured non-integrated patient support comprising the positioned patient; and deciding 460 if the configuration of the configured integrated patient support comprising the patient is appropriate to position the patient correctly for medical imaging and/or radiotherapy (461) or if the configuration of the configured integrated patient support comprising the patient is not appropriate to position the patient correctly for medical imaging and/or radiotherapy (462). In some embodiments, checking 450 the configuration of the configured integrated patient support comprising the positioned patient comprises a clinician visually checking the configuration. In some embodiments, checking 450 the configuration of the configured integrated patient support comprising the positioned patient comprises comparing an image of the configured integrated patient support with a reference image of a patient support in a correct configuration. In some embodiments, checking 450 the configuration of the configured non integrated patient support comprising the positioned patient comprises comparing one or more index values for a number of components of the patient support with one or more reference index values of a patient support in a correct configuration. In some embodiments, checking 450 the configuration of the configured integrated patient support comprising the positioned patient comprises simulating medical imaging (e.g., simulating a CT scan) or simulating radiotherapy of the patient.

If the deciding step (460) identifies the configured integrated patient support as not being appropriate to position the patient correctly for medical imaging and/or radiotherapy (462), then methods 400 comprise adjusting 465 the configuration of the configured integrated patient support comprising the positioned patient; and repeating the steps of checking 450 the configuration of the configured integrated patient support comprising the positioned patient; and deciding 460 if the configuration of the configured integrated patient support comprising the patient is appropriate to position the patient correctly for medical imaging and/or radiotherapy (461) or if the configuration of the configured integrated patient support comprising the patient is not appropriate to position the patient correctly for medical imaging and/or radiotherapy (462). After performing a number of checking 450 and deciding 460 steps, and performing a number of adjusting 465 step(s) if needed, to identify or verify that the configuration of the configured integrated patient support comprising the patient is appropriate to position the patient correctly for medical imaging and/or radiotherapy (461), then method 400 comprises performing 470 medical imaging and/or radiotherapy on the patient.

In some embodiments of method 400, checking and deciding steps, and adjusting step(s) if needed, are performed after configuring 430 the integrated patient support and prior to positioning the patient on the configured integrated patient support.

Thus, in some embodiments, the technology provides methods comprising an embodiment of method 300 and an embodiment of method 400 performed as one method comprising steps of method 300 and steps of method 400.

In some embodiments, steps of the described methods (e.g., method 300 and/or method 400) are implemented in software code, e.g., a series of procedural steps instructing a computer and/or a microprocessor to produce and/or transform data; to energize and/or activate a motor to translate and/or rotate a component of a patient support; and/or to store or retrieve data (e.g., patient data or a patient support configuration) from a tangible medium as described above. In some embodiments, software instructions are encoded in a programming language such as, e.g., BASIC, Java, C, C++, C#, Python, PHP, Ruby, Perl, MATLAB, Mathematica, Object Pascal, Objective-C, Swift, Scala, Common Lisp, and Smalltalk.

In some embodiments, one or more steps are provided in individual software objects connected in a modular system. In some embodiments, the software objects are extensible and portable. In some embodiments, the objects comprise data structures and operations that transform the object data. In some embodiments, the objects are used by manipulating their data and invoking their methods. Accordingly, embodiments provide software objects that imitate, model, or provide concrete entities, e.g., for numbers, shapes, data structures, that are manipulable. In some embodiments, software objects are operational in a computer or in a microprocessor. In some embodiments, software objects are stored on a computer readable medium.

In some embodiments, a step of a method described herein is provided as an object method. In some embodiments, data and/or a data structure described herein is provided as an object data structure.

Some embodiments provide an object-oriented pipeline for performing an embodiment of method 300, an embodiment of method 400, or a method comprising a combination of method 300 followed by method 400, e.g., comprising one or more software objects, to record a patient support configuration (e.g., for a non-integrated patient support) and configure a patient support (e.g., an integrated patient support) using the recorded patient support configuration.

Embodiments comprise use of code that produces and manipulates software objects, e.g., as encoded using a language such as but not limited to BASIC, Java, C, C++, C#, Python, PHP, Ruby, Perl, MATLAB, Mathematica, Object Pascal, Objective-C, Swift, Scala, Common Lisp, and Smalltalk.

Although the disclosure herein refers to certain illustrated embodiments, it is to be understood that these embodiments are presented by way of example and not by way of limitation. All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1-47. (canceled)

48. A medical therapy system comprising:
a subsystem comprising a first patient support; and
a second patient support.

49. The medical therapy system of claim 48, wherein the first patient support is structured to support a patient in an upright position and the second patient support is structured to support the patient in the upright position.

50. The medical therapy system of claim 49, wherein the upright position is a standing, sitting, kneeling, or perched position.

51. The medical therapy system of claim 48, wherein the subsystem is a medical imaging subsystem comprising an imaging source and a detector.

52. The medical therapy system of claim 48, wherein the subsystem is a radiotherapy subsystem comprising a radiotherapy source.

53. The medical therapy system of claim 48, wherein the subsystem is a medical imaging and radiotherapy subsystem comprising an imaging source, a detector, and a radiotherapy source.

54. The medical therapy system of claim 53, wherein the radiotherapy source is a static source.

55. The medical therapy system of claim 48, wherein the first patient support is configured to rotate around a substantially vertical axis.

56. The medical therapy system of claim 48, wherein the first patient support is coupled to components that translate the first patient support in three dimensions and that rotate the first patient support around three axes.

57. The medical therapy system of claim 48, wherein the second patient support comprises a stand or base.

58. The medical therapy system of claim 48, wherein the second patient support is mounted to a floor.

59. The medical therapy system of claim 48, wherein the first patient support and second patient support are in different rooms.

60. The medical therapy system of claim 48, further comprising a database.

61. The medical therapy system of claim 60, further comprising a computer.

62. The medical therapy system of claim 60, wherein the database comprises a data structure describing a patient support configuration comprising a number of index values recorded from the second patient support.

63. The medical therapy system of claim 62, wherein the database further comprises patient information for a patient and wherein the patient information is associated with a patient support configuration.

64. The medical therapy system of claim 62, wherein the index values comprise a back rest height index value, a back rest angle index value, a seat pan height index value, a seat pan angle index value, a shin rest distance index value, a shin rest height index value, a shin rest angle index value, a foot brace or heel stop distance index value, an arm rest height index value, an arm rest arm angle index value, and/or an arm rest bent portion angle index value.

65. The medical therapy system of claim 63, wherein the first patient support is configured according to the patient support configuration for the patient.

66. The medical therapy system of claim 65, wherein the patient is identified or associated with the patient information.

67. The medical therapy system of claim 48, wherein the first patient support comprises a seat pan, a shin rest, and a heel stop; and wherein the second patient support comprises a seat pan, a shin rest, and a heel stop.

68. The medical therapy system of claim 48, wherein the first patient support further comprises a back rest; and wherein the second patient support further comprises a back rest.
